(19) United States
(12) Patent Application Publication
(10) Pub. No.: US 2026/0248640 A1
(43) Pub. Date: Aug. 27, 2026

(54) Removable Airway Appliance

(71) Applicant: Nathan Milo Tanner, Medford, OR (US)

(72) Inventor: Nathan Milo Tanner, Medford, OR (US)

(21) Appl. No.: 19/653,876

(22) Filed: Apr. 21, 2026

Related U.S. Application Data
(63) Continuation-in-part of application No. 18/154,996, filed on Jan. 16, 2023, now abandoned.
(60) Provisional application No. 63/300,090, filed on Jan. 17, 2022.

(51) Int. Cl. A61F 5/56; A61F 5/058

(52) U.S. Cl. CPC A61F 5/566; A61F 5/05891

(57) ABSTRACT

Disclosed is a removable airway appliance for guiding and developing the lower and upper jaws forward. The removable airway appliance may include an anterior acrylic section having a specified amount of the palate/roof of the mouth and lingual side/inside of top front teeth to direct the forces of development, a posterior acrylic section providing retention over the primary/baby molars, expansion screws set at different angles for up/down and side/side growth and development, and clasps for retention in case if permanent teeth are present. On the horizontal axis the screws can be positioned flat at a small angle or at a steeper angle to help direct the direction of growth and development of the upper and lower jawbones, while the side-to-side directs the amount of increased width of the arch.

### CROSS REFERENCE TO RELATED APPLICATIONS

The present application is a continuation-in-part of U.S. patent application Ser. No. 18/154,996, filed Jan. 16, 2023, which claims the benefit of the filing date of U.S. Provisional Patent Application Ser. No. 63/300,090, filed Jan. 17, 2022. The foregoing applications are incorporated by reference in their entireties as if fully set forth herein.

### TECHNICAL FIELD

The present disclosure relates generally to a removable airway appliance. More specifically, and without limitation, the present disclosure relates to a removable airway appliance for developing the bone of the upper and lower jaws forward permanently. However, the present disclosure is not limited to these novel and inventive systems and improvements and methods of use, and it may further be adapted for a variety of purposes.

### BACKGROUND

Sleep disorders are a group of conditions that affect the ability to sleep well on a regular basis. Sleep disorders are caused by too much soft tissue in the throat that leads to collapsing of the airway. Most people occasionally experience sleeping problems due to stress, hectic schedules, and other outside influences. However, when these issues begin to occur on a regular basis and interfere with daily life, they may indicate a sleeping disorder. Depending on the type of sleep disorder, people may have a difficult time falling asleep and may feel extremely tired throughout the day. The lack of sleep can have a negative impact on energy, mood, concentration, and overall health. In some cases, sleep disorders can be a symptom of another medical or mental health condition. These sleeping problems may eventually go away once treatment is obtained for the underlying cause. When sleep disorders aren't caused by another condition, treatment normally involves a combination of medical treatments and lifestyle changes.

There are many different types of sleep disorders like Insomnia, Sleep apnea, Parasomnias, Restless leg syndrome, Narcolepsy, and (UARS) Upper Airway Respiratory Syndrome. Some may be caused by other underlying health conditions. Sleep apnea is a common sleep disorder. It occurs when the muscles in the back of the person's throat relax and then narrow or close the airway. With the tissue blocking the air passage, the person can't get air in and air can't get out. During sleep apnea, the person repeatedly stops breathing during sleep. The person will briefly wake up to resume breathing, even if that particular person isn't aware of it. The interrupted sleep can lead to symptoms such as: excessive daytime sleepiness; snoring; sleep deprivation; insomnia; dry mouth; headache.

Sleep apnea is a potentially serious sleep disorder in which breathing repeatedly stops and starts. Snoring, upper airway resistance syndrome and obstructive sleep apnea syndrome (OSAS) are all related to narrowing or obstruction of the upper airway during sleep (sleep disordered breathing). According to the national institutes of health (NIH), approximately 18 million. Americans have sleep apnea (sleep disordered breathing), but fewer than 50% are presently being diagnosed. According to the national highway traffic and safety administration (NHTSA), 100,000 accidents and 1,500 traffic fatalities per year are related to drowsy driving. More than 50 percent, of Americans over age 65 have sleep difficulties, and prevalence of sleep problems will therefore increase as the over-65 population increases. Each year, sleep disorders, sleep deprivation, and excessive daytime sleepiness add approximately $16 billion annually to the cost of health care in the U.S. and result in $50 billion annually in lost productivity.

The upper airway (air filled space between the nose and larynx) of human beings is curvy, so it is more flexible than that of other species and is more prone to collapse under negative pressure. The air cavity at the back of the throat is partly responsible for sleep disorders. When human beings are awake, a certain amount of tone is present in upper airway muscles to prevent this collapse. During sleep time, muscle tone decreases in upper airway muscles and in this way, in some individuals, this causes the airway to collapse.

A small amount of decreased airflow or brief obstructions occur in all humans during sleep. They are counted as medically significant if airflow is decreased more than 30% of normal (hypopnea) or if airflow is obstructed for more than 10 seconds (apnea) for the severity of the sleep disorder. These repetitions of hypopnea or apnea cause some degree of arousal during sleep. The patient is not always completely awakened to full consciousness, the sleep pattern is disturbed causing the patient to feel sleepy during the day. If the frequency of hypopnea or apnea is less than 5 times an hour it is called upper airway resistance syndrome. These patients often show symptoms related to sleep disruption. If the frequency is more than 5 times an hour it is sleep apnea.

Studies are now demonstrating the detrimental impact of fragmented and disrupted sleep cycles. Various experiments have been carried out to expand the upper and lower jaws in a forward direction. In the 1950's Dr. John Mew In England started using orthotropic using a Bio Block stage 1 to expand the upper jaw and a Bio Block stage 2 to force the lower jaw forward. The upper teeth would be flared out and the lower jaw was forced forward with the Bio Block stage 2, and in 1980's and 90's Dr. Bill Hang in California developed his own practices that improved on the Mew invention, he calls it an Expanser on the top, it also flares the upper teeth, and he calls the lower appliance the Adapt Appliance and it forces the lower jaw forward. Dr. Bill Hang also does reopening extraction spaces to make more room for the tongue in the mouth.

There are various Mandibular (jaw) advancement devices (MAD's) and jaw development devices. The Adjustable PM Positioner was invented by Dr. Jonathan A. Parker. Its unique design is durable, comfortable, and effective. The Adjustable PM Positioner is constructed of a heat-sensitive acrylic (Bruxeze) that fits comfortably over the upper and lower teeth. The appliance stabilizes the jaw position by allowing only a small amount of jaw movement. The adjustment mechanisms are on the cheek side of the appliance and allow for easy advancement of the jaw position to improve effectiveness of the device when it is indicated. It is a durable and effective option.

One of the prior art means is disclosed in patent number 20210007831 as “A Dental system for symmetry of jaw, palate and teeth”. A dental apparatus, systems and methods for providing anterior, lateral and vertical movement, an apparatus including a posterior portion to be fixed to and supported by a posterior dental structure of a patient; and an anterior portion connected to and separable from the posterior portion, the anterior portion operable to provide anterior movement, lateral movement and vertical movement to an anterior dental structure of the patient. The dental system include screws that can be positioned anywhere in the device for controlled movement in multiple directions (planes) for dental structure movement in adults and children. The adjustable dental expander devices provide anterior, vertical, and/or anterior-lateral movement to teeth, a jawline, and/or palate. The devices are compact and reduce bulkiness and provide a soft surface to reduce direct pressure and allow for 24-hour wear and slow controlled movement. The devices do not impede speech while worn. One or more surfaces of the device is optionally covered with a thin cover or coating of soft, gel like material to distribute pressure and guard against painful pressure points. A suitable gel-like material is soft to prevent undue pressure on any particular point, but provides gentle pressure over time at particular points.

The device comprises of a hinge and a wire in between first anterior portion and first posterior portion and a second adjustable and lockable hinge is present in between second anterior portion and second posterior portion. The device includes a screw base and a screw which is operable to allow rotation in and out of the screw base such that the rotation provides movement and pressure in the anterior dental structure. The device includes one or more attachable pads positioned on the anterior portion of the device which provides movement and pressure to the anterior dental structure. Dental device can be inserted to be made semi-permanent via dental cementation or glue, or it can be removable and re-insertable by the patient and the doctor. The device can be applied for movement in the upper portions and lower portions of a patient's mouth to apply movement to upper and lower jaw structure.

The posterior and anterior portion are connected by a wire, which is operable to move in an anterior direction and hinge is allowing rotation of the anterior portion.

Another one prior art means is disclosed in patent no. 20200214804 as Orthodontic appliance. The present invention shows a jaw shape modifying device comprising a frontal shell portion and lateral shell portions connected by adjustment mechanisms for adjusting the frontal distance between the frontal shell portion and the lateral shell portions. It is comprised of a shell shaped structure which includes a shape-biasing region which extends beyond the patient's internal gum line when the shell is worn. The appliance reduces the severity of orthodontic relapse, thereby achieving a more stable outcome for the patient.

The shell includes a front shell secured to lateral shell portions by way of an adjustment mechanism for adjusting a distance between the frontal shell portion and lateral shell portions by way of an adjustment mechanism for adjusting a distance between the frontal shell portions and the lateral shell portions. The forward region of the patient's jaw is extended by increasing the frontal distance over time. The adjustment mechanism includes an expansion screw securable to the jaw basing region of each shell portion. The shell portions are interconnected to one another by one or more expansion screws which are easy to operate.

The appliance comprises one or more anchor points to which a resilient band is attachable. An anchor point could be a hook embedded into the appliance. The first appliance is configured to be worn over the patient's upper teeth, and the second appliance is configured to be worn over the patient's lower teeth. The orthodontic appliances modify the shape of one or more jaws individually but also work to alter the position of one jaw relative to the other.

Another prior art means is disclosed in U.S. Pat. No. 5,002,485 as “orthopedic appliance”. It shows an orthopedic appliance having a universal screw assembly that interconnects frontal portions and the first and second side portions and operates to independently adjust the lateral spacing of the side portions from one another and the anterior-posterior spacing of the frontal portion from the side portions. The present invention is an orthopedic appliance for correcting class II malocclusions comprises a frontal portion to engage the mandibular and the maxillary frontal arches and first and second side portions, posterior to the frontal portion. Configured to engage at least some of the maxillary molars. A universal screw assembly interconnects the frontal portions and the first and second side portions and operate to adjust the lateral spacing of the side portions from one another and the anterior posterior spacing of the frontal portion from the side portions. The appliance is expanded in stages to maximize the utilization of corrective lower jaw movements which results from securing the appliance in patient's upper mouth.

Another prior art means is disclosed in U.S. Pat. No. 4,433,956 as “Orthopedic corrector and method of correction of class II malocclusion”. The orthopedic appliance is designed so that when the posterior and anterior segments are minimally expanded, the mandibular-maxilla position is changed from which exists prior to treatment. As time progresses, the expansion screw assemblies are turned, separating the anterior and posterior segments with resultant movement of the mandible more forward. The orthopedic corrector of the appliance is outfitted with orthodontic attachments such as a labial archwire. An improved orthopedic appliance for correcting a class II malocclusion comprising an acrylic anterior segment molded to fit the lower mouth and dentition and two acrylic posterior segments molded to fit upper mouth and dentition of a patient and an expansion screw connecting each posterior segment to the anterior segment for expandable movement between the anterior segment and the posterior segments. The appliance is expanded in stages to maximize the utilization of corrective lower jaw movements which results from the anchoring of the orthopedic appliance in the patient's upper mouth.

Another one prior art means is disclosed in U.S. Pat. No. 4,431,411 as “Segmented labial bow with lip pads for aid in the correction of class III malocclusions and malocclusions with underdeveloped maxillas”. The segmented labial bow with lip pads is attached to a conventional orthopedic/orthodontic appliance. As the orthopedic/orthodontic appliance is activated, the lip pads on the segmented bow hold the upper lip away from the roots of the maxillary anterior teeth and alveolar process allowing skeletal development of the premaxillary area with resultant forward movement of the roots and aveolar bone of the premaxillary teeth. It shows an acrylic orthopedic/orthodontic appliance having clasps 12, 15 for attachment to the teeth and jackscrews 16 that cause the premaxilla to move forward in relation to the mandible.

The segmented bows are attached to the sides of the active orthopedic appliance with solder on a wire originating in the acrylic portion of the appliance.

Another prior art means is disclosed in KR 20210008908 as mouthpiece. There is provided a mouthpiece, characterized in that the divided pieces are overlapped so that a surface that faces or comes into contact with the skin of the oral cavity does not form a gap despite the relative movement or approach of the divided pieces.

The mouthpiece of the present invention for solving the above problems is mounted in the oral cavity and used to reform the shape of the skull including the maxilla, and consists of a plurality of divided pieces, at least some of the divided pieces are formed by a screw. In the mouthpiece configured to be approached and spaced from each other, the divided pieces are overlapped so that the surfaces of the divided pieces that face or come into contact with the skin in the oral cavity do not form a gap despite the relative movement or approach of the divided pieces. It is mounted in the oral cavity and used to modify the shape of the skull including the maxilla, and is composed of a plurality of divided pieces, wherein at least some of the divided pieces are configured to be approached and spaced apart from each other by a screw, the mouthpiece comprising: At least some of the dividing pieces are connected to a headgear mounted outside the oral cavity, and the reaction force vector with respect to the action force vector by the screw is set to be offset by the support force vector by the headgear.

Or, it is mounted in the oral cavity and used to modify the shape of the skull including the maxilla, and is composed of a plurality of divided pieces, wherein at least some of the divided pieces are configured to be approached and spaced apart from each other by a screw, the mouthpiece comprising: At least some of the split pieces are connected to a connecting wire, the connecting wire is connected to a headgear mounted outside the oral cavity, and a blind hole or a through hole is formed in the latch on the lower surface of the split piece connected to the connecting wire. The hook of the connecting wire is characterized in that it is provided with a projection (positive shape) that is detachably coupled to the blind hole or the through hole.

Or, it is mounted in the oral cavity and used to modify the shape of the skull including the maxilla, and is composed of a plurality of divided pieces, wherein at least some of the divided pieces are configured to be approached and spaced apart from each other by a screw, the mouthpiece comprising: At least one of the divided pieces is preferably provided with a power storage device for storing electrical energy or a power generation device for converting the oral masticatory force into electrical energy by the piezoelectric phenomenon.

It is characterized in that it is made of a material having a surface roughness Ra of 0.2 to 0.4 so as not to form a bacterial film.

Another prior art means is disclosed in KR 970010577 as device for jaw orthopedic. The Han appliance fundamentally treats the third class malocclusion of jaw bones without any operation by effectively utilizing the period of most active growth orthopedically to induce the proper growth of the bones. The construction includes a premaxillae (500), an orthopedic part (100) tightly attached to the inner side of a premaxillae (500), q fixation part (200) fixed to the inner side of the upper jaw molar teeth and palatal surface, the parts (100) and (200) having a jack screw (300) installed between them, the fixation part (200) being connected to a lower jaw holder (400) on the inner side of the lower jaw bone to be integral, the fixation part (200) and the holder (40) having a protrusive bite block (30) for pressure-attaching the lower and upper molar teeth (502, 602) on both sides of the loader center. The holder (40) having a labial bow (41) on its tip to fix the lower anterior teeth.

Another prior art means is disclosed in WO2021049673 as a “Mouthpiece”. Provided is a mouthpiece which is mounted in an oral cavity for use in changing the shape of the skull including the maxillary bone and is composed of a plurality of segment pieces, at least some of the segment pieces being configured to approach or be spaced apart from each other by means of a screw, wherein the surfaces of the segment pieces facing or contacting the skin in the oral cavity are configured such that the segment pieces overlap with each other, such that a gap does not occur in spite of the approaching or spacing-apart movement of the segment pieces relative to each other.

Or, it is mounted in the oral cavity and used to modify the shape of the skull including the maxilla, and is composed of a plurality of divided pieces, wherein at least some of the divided pieces are configured to be approached and spaced apart from each other by a screw, the mouthpiece comprising: At least some of the dividing pieces are connected to a headgear mounted outside the oral cavity, and a reaction force vector with respect to an action force vector by the screw is set to be offset by a support force vector by the headgear.

Or, it is mounted in the oral cavity and used to modify the shape of the skull including the maxilla, and is composed of a plurality of divided pieces, wherein at least sonic of the divided pieces are configured to be approached and spaced apart from each other by a screw, the mouthpiece comprising: At least some of the divided piece, the connecting wire is connected to the connecting wire is connected to a headgear mounted in addition to the oral cavity, the latch of the lower face of the split pieces are connected to the connecting wire, blind holes or through holes are formed, The hook of the connecting wire is characterized in that it is provided with a projection (positive shape) that is detachably coupled to the blind hole or the through hole.

Or, it is mounted in the oral cavity and used to modify the shape of the skull including the maxilla, and is composed of a plurality of divided pieces, wherein at least some of the divided pieces are configured to be approached and spaced apart from each other by a screw, the mouthpiece comprising: At least one of the divided pieces is preferably provided with a power storage device for storing electrical energy or a power generation device for converting the oral masticatory force into electrical energy by the piezoelectric phenomenon.

At this time, it is preferable that at least one of a low-frequency generator, an infrared LED, an ultraviolet LED, a screw driving motor, a wireless communication module with an external terminal, and a screw operation amount sensor is driven by the electric energy.

In addition, it is characterized in that it is made of a material having a surface roughness Ra of 0.2 to 0.4 so as not to form a bacterial film.

Another prior art means is disclosed in patent no: EP2067452 as “Orthodontic device”. Orthodontic device (1) for activating the growth of the lower jaw for use in combination with fixed orthodontic appliances, comprising two plastic bodies (2,2′), each provided with at least one counter-surface corresponding to the chewing surface of molars in the upper jaw (6,6′), and at least one counter-surface corresponding to the chewing surface of molars in the lower jaw (7,7′), and further attaching means (8, 8′, 9, 9′) for attaching to the upper jaw, characterized in that each plastic body (2, 2′) comprises at least two parts ((4, 5), (4′, 5′)) which can be displaced and fixed relative to each other substantially parallel to the longitudinal direction of the respective counter-surfaces, wherein each part is provided with at least one of said counter-surfaces.

### Dorsal (Somnodent)

The Dorsal appliance can be made of a hard acrylic or an acrylic material that has a softer inner lining It is specifically designed with separate upper and lower portions for easier placement and removal. There is a wing on the lower portion and an acrylic block on the upper portion that encases a screw mechanism for easy advancement. This is a good choice for patients with smaller mouths or narrower dental arches and is a comfortable and effective choice for sleep apnea.

### Dream TAP

The Dream TAP is made of a material that has a hard outer shell and softer inner lining It has a hook and screw on the front of the lower portion and the hook attaches to a wire on the upper portion that connects the appliance together. Since the screw mechanism is on the front of the device, the appliance can be advanced while in the mouth by using a small key. It is durable and effective in treating sleep apnea.

### EMA

The EMA is made of thinner acrylic material that fits over the teeth and the upper and lower portions are connected by flexible straps. Straps of different lengths and flexibilities allow for advancing the jaw position gradually and they allow freedom of movement of the jaw. This device is used in patients with primary snoring and milder sleep apnea conditions.

### Herbst

The Herbst appliance is made of either a heat-sensitive or hard acrylic material and has a piston-type (post within a tube) screw mechanism along the sides of the device. It allows good freedom of movement of the jaw in all directions. It is durable and effective in treating sleep apnea.

### Micro2

The Micro2 is an all-acrylic (no metal) appliance that has wings on the upper and lower portions that are positioned against each other to hold the lower jaw forward. The design provides a lower profile and allows for good jaw movement. The patient receives 4-6 units that have the wings positioned in a way that allows for mixing and matching these units to gradually advance the jaw position. This device is an effective option for treating sleep apnea.

### Narval

The Narval is made of a nylon material that allows it to be one of the thinnest and lightest appliances available. It has plastic straps that connect the upper and lower portions and has no metal components. It allows for good jaw movement, is effective for treating sleep apnea, and is a good option for patients who have a smaller mouth.

### SUAD

The SUAD appliance is made of a comfortable heat-sensitive acrylic and is reinforced by a metal framework. The adjustment mechanism (Herbst-type) is positioned on the sides of the device and allows good freedom of movement of the jaw. This appliance is a frequent choice for patients who tend to grind their teeth heavily. Adjustable pm positioner; Dorsal (somnodent); Dream tap; Ema; Herbst; Micro2; Narval; Suad are some mandibular (jaw) advancement appliances which allows the freedom for good movement of jaw.

The inefficiency of the orthotropic appliances is that they can only be applied on ages 7-11 years old. If they are not started in between age 7-11, they can not be done. The deficiencies are that the age for treatment is very limited to age 7-11 yrs old only. Also, it flares the upper teeth way out, and forces the lower jaw forward. Most of the sleep appliances and MAD appliances are night time only appliances and do not permanently change or develop the jaw structures. They help to open the airway during sleeping only at night. The tongue is still trapped behind the retruded upper jaw because night-time sleep appliances only move the lower jaw forward. Also over time there are increasing cases of the Maxilla moving backward over time.

The following are the drawbacks of the prior arts:
1. Some of these products that develop the upper and lower jaws forward can only be done after age 6 when the first permanent molars are fully erupted into the mouth.
2. The age for treatment for some products is very limited to age 7-11 yrs old only. Also, treatment flares the upper teeth way out, and forces the lower jaw forward, which is a difficult and uncomfortable process. Very few dentists and patients attempt this.
3. Most of the sleep appliances and MAD appliances are nighttime only appliances and do not permanently change or develop the jaw structures. They help to open the airway during sleeping only at night by moving the lower jaw forward temporarily and there is a distalizing force on this upper jaw.
4. The oral sleep appliances support the lower jaw in a more forward position moving the back of the tongue forward as well, but the front of the tongue is still restricted by the front teeth of the upper jaw which is commonly underdeveloped.
5. There are no appliances that can be used to develop the upper and lower jaws forward in the all primary dentition (all baby teeth with no permanent teeth in yet, ages 2-6).
6. There are no appliances that can be used to develop the upper and lower jaws forward in the early primary dentition with only 16 baby teeth erupted into the mouth (ages 18 months to two and a half years of age).
7. No prior art describes changing the vertical angle of the screw in precise increments to reverse excess vertical or downward growth.
8. No prior art describes embodiments that cover the canines to maintain the canine roots in a vertical orientation while developing the front of the upper jaw upward and forward in a controlled fashion.
9. No prior art describes two support wires to improve strength and durability of the removable acrylic appliances.
10. No prior art is able to predictably reverse a gummy smile or excess downward jaw growth non surgically in a large variety of age groups.

So, there is a need for a removable airway appliance that develops the jaw bone forward, guiding the lower jaw bone to grow forward with the upper jaw bone, getting the forward jaw growth of the upper and lower jaws, starting at any age, even at age 2 all the way to advanced adulthood. The present invention is used in all primary dentition (all baby teeth) and permanent teeth for people from age 2-to older adult and can be used in a one year old with only 16 baby teeth. The present invention has the ability to reverse excess vertical/downward growth (controlling the amount and direction of growth/development) and has the ability to guide growing jaws to improve airway (covering canines) or making room for teeth (acrylic behind canines). Smooth surface over the biting area of the teeth help to stimulate the forward growth of the lower jaw and creates a more open airway to breathe better and to reduce or eliminate sleep apnea. The present invention helps to grow and develop the jaw structures permanently in a more forward and ideal position. The present invention helps develop the bone of the upper and lower jaws forward permanently.

Thus, the present disclosure provides the state of the art with a novel, removable airway appliance in the Maxilla, comprising an acrylic base molded over certain portions of the upper dentition wherein the base includes one or more screw adjustment members operable to translate a section of the base in an upward and/or forward direction, creating more space in the mouth for the tongue by developing the bone of the upper and lower jaws forward permanently and a method of use utilizing wire clasps may also be used to secure the device to the dentition. As more space is developed in the mouth for the tongue, there is also more space at the back of the throat for airflow and breathing because the tongue is in a more forward position.

### OBJECTIVES

The main objective of the present invention is to remove all the associated drawbacks and to provide a novel, a removable airway appliance for developing the bone of the upper and lower jaws forward permanently and a method of use.

Yet another object of the present invention is to guide and develop the lower and upper jaws forward, also widen the upper jaw, making room for the tongue in the mouth to maximize the airway space by the precise angulation of the screws.

Yet another object of the present invention, for young children ages 1-6, is to prevent crowding of the teeth and to make room for all permanent teeth to come into the mouth without crowding. Creating space for all the permanent teeth before they come in and prevent crowding, especially useful for young children to avoid future crowding.

Yet another object of the present invention is to develop the upper and lower jaws forward to get the back of the tongue forward away from the back of the throat which improves airflow for breathing and sleeping.

Yet another objective of the present invention is to help in growth of the upper jaw forward and unrestrict the lower jaw that is often trapped back behind the upper jaw.

Another objective of the present invention is to provide a removable airway appliance which provides better airway, better nasal breathing, upper and lower jaw forward growth, provides better cheek structure and better lip support to the human face.

Another object of the present invention is to wear the appliance 20-24 hours a day only removing a few minutes a day to brush for younger children (age 1-13) and worn 20-22 hours a day for older teenagers and adults age 14+.

Another object of the present invention is to Use the removable airway appliance in all primary dentition (all baby teeth) and permanent teeth for people from age 2-to older adult.

Another object of the present invention is that it can be used with only 16 baby teeth.

Another object of the present invention is to reverse excess vertical/downward growth (controlling the amount and direction of growth/development) by using the removable airway appliance by the vertical angulation of the screws. In this way it can help to reverse an excessively gummy smile.

Another object of the present invention is to control growing jaws to improve airway (covering canines) or making room for teeth (acrylic behind canines).

Another object of the present invention is to provide a smooth surface over the biting area of the teeth and help to stimulate the forward growth of the lower jaw.

Another object of the present invention is to create a more open airway to breathe better and to reduce or eliminate sleep apnea.

Another object of the present invention is to expand (dental arches) the crescent arrangement of the patient's teeth. Even in cases of severe crowding in children or adults, no permanent teeth need to be removed to develop the jaws, open the airway and straighten the teeth.

### SUMMARY

The present disclosure provides the state of the art with a novel removable airway appliance for guiding and developing the lower and upper jaws forward. Furthermore, the present disclosure provides a removable airway appliance for guiding and developing the lower and upper jaws forward. The present invention prevents crowding of the teeth and makes room for all the teeth, making room for all permanent teeth without crowding, creates space for all the permanent teeth. The present invention is that it may grow the upper jaw forward and unrestrict the lower jaw that is often trapped back behind the upper jaw. The present invention guides and develops the lower and upper jaws forward, and widen the upper jaw, making room for the tongue in the mouth to maximize the airway space. In the removable airway appliance, on the horizontal axis the screws can be positioned flat at a small angle or at a steeper angle to help direct the direction of growth and development of the jaw bone. The side angle is directing the amount of increased width of the arch.

Systems that embody the disclosure, in accordance with the aspects thereof, are typically designed by describing their functions. Therefore, the present disclosure is also embodied in such descriptions, and methods of describing systems, but the scope of the present disclosure is not limited thereby. Therefore, these and other objects of the disclosure are hereby considered, not only those limited to those found in this summary of the disclosure section, but as can be found throughout the scope of this specification and accompanying documents.

The details of one or more embodiments of the subject matter described in this specification are set forth in the accompanying drawings and the description below. Other features, aspects, and advantages of the subject matter will become apparent from the description, the drawings, and the claims.

Those skilled in the art will appreciate that the conception upon which this disclosure is based readily may be utilized as a basis for the designing of other structures, methods and systems that include one or more of the various features described below.

Certain terminology and derivations thereof may be used in the following description for convenience in reference only, and will not be limiting. For example, words such as “upward,” “downward,” “left,” and “right” would refer to directions in the drawings to which reference is made unless otherwise stated. Similarly, words such as “inward” and “outward” would refer to directions toward and away from, respectively, the geometric center of a device or area and designated parts thereof. References in the singular tense include the plural, and vice versa, unless otherwise noted.

### BRIEF DESCRIPTION OF THE DRAWINGS

FIG. 1 is a plan view of an appliance from a vantage looking up into a child's upper mouth and palate with the appliance over the back baby molars and sitting behind the front teeth;

FIG. 2 is a side view of a clasped upper appliance;

FIG. 3 is a front view of a claspless appliance;

FIGS. 4-6 are bottom views of a claspless appliance leaning up against a model of a mouth;

FIG. 7 is a bottom view of a claspless appliance showing the biting surface;

FIG. 8 is a bottom view of a claspless appliance leaning up against a model of a mouth;

FIG. 9 is a plan view of a clasped appliance;

FIG. 10 is a plan view of a clasped appliance on a model of a mouth;

FIG. 11 is a plan view of a front four claspless appliance;

FIGS. 12A and 12B are schematic top views of an appliance demonstrating lateral angulation/side to side/width; and

FIGS. 13A and 13B are schematic side views of an appliance demonstrating vertical angulation/up down.

### DETAILED DESCRIPTION

In the following detailed description, reference is made to the accompanying drawings which form a part hereof, and in which is shown by way of illustration specific embodiments in which the disclosure may be practiced. These embodiments are described in sufficient detail to enable those skilled in the art to practice the disclosure, and it is to be understood that other embodiments may be utilized and that mechanical, procedural and other changes may be made without departing from the spirit and scope of the disclosure. The following detailed description is, therefore, not to be taken in a limiting sense, and the scope of the disclosure is defined only by the appended claims, along with the full scope of equivalents to which such claims are entitled.

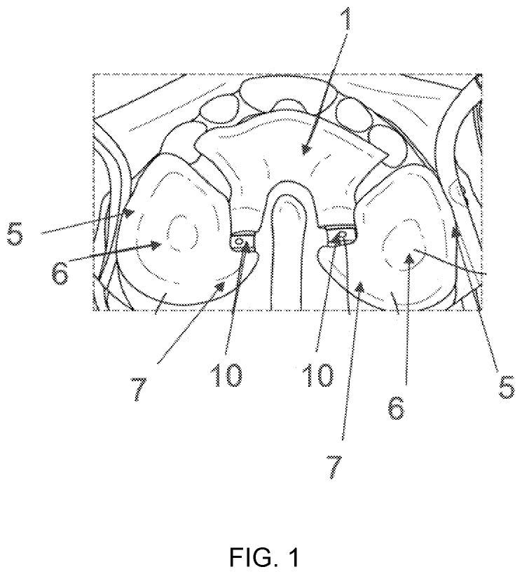

Referring to FIGS. 1 through 11, wherein like reference numerals refer to like components in the various views, there is illustrated therein a removable airway appliance, generally denominated 100 herein.

### REFERENCE NUMERALS

1. Front acrylic section against front palate, gum tissue and the back side of the front teeth.
2. Support wire on the inside of the acrylic in the front section of acrylic.
3. Lingual on canines, acrylic is only on the lingual (inside or tongue side) surface of the upper canines. Acrylic near canines is connected to the acrylic in the front of the mouth.
4. Distal on canines, acrylic is over the distal (back side), facial (front side) and lingual (inside or tongue side) of the upper canines. Acrylic around the canines is connected to the acrylic in the front of the mouth.
5. Acrylic on the outside of the baby molar teeth on the claspless appliance design.
6. Acrylic covering the biting surface of the back teeth (molar teeth).
7. Acrylic on the inside of the mouth on the side of the palate next to the molars.
8. Clasp on the back teeth for the clasp design style appliances.
9. Screw placed into acrylic at 3 degree vertical and widening angle.
10. Screw placed into acrylic at 5 degree vertical and ridge angle.
11. Screw placed into acrylic at 10 degree vertical angle and straight or parallel position.
12. Screw placed into acrylic at 15 degree vertical and widening angles.
13. Screw placed into acrylic at 20 degree vertical and ridge angle.
14. Acrylic completely over canines and the acrylic over the canines is connected to the acrylic in the back of the mouth by the insertion of a screw assembly joining the front and back acrylic sections.
100. Removable airway appliance.

FIG. 1 is a plan view of an appliance 100 looking up into a child's upper mouth and palate with the appliance over the back baby molars and sitting behind the front teeth. The appliance includes front acrylic section 1 against the front palate, gum tissue and the back side of the front teeth, acrylic on the outside of the baby molar teeth 5, acrylic covering the biting surface of the back teeth 6, acrylic on the inside of the mouth on the side of the palate next to the molars 7, and screws 10 placed into the acrylic at 5 degree vertical and ridge angles. The claspless appliance is only for children ages 2-6.

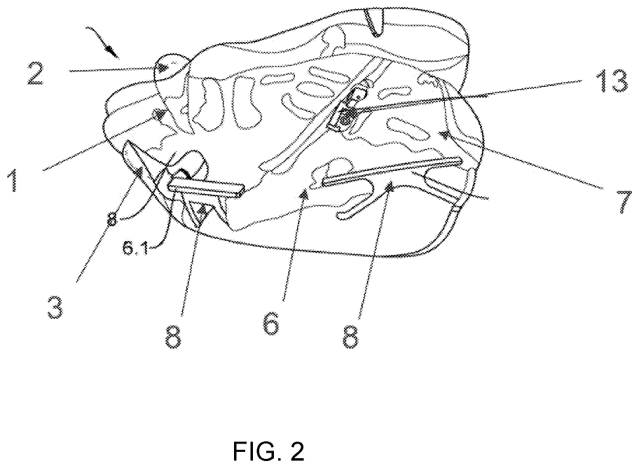

FIG. 2 is a side view of a clasped upper appliance 100. The part toward the top of the drawing is what would go up against the palate, teeth and gums. The appliance includes front acrylic section 1 against the front palate, gum tissue and the back side of the front teeth, support wire 2 on the inside of the acrylic in the front section of acrylic, lingual on canines acrylic 3 only on the lingual surface of the upper canines, the acrylic near the canines connected to the acrylic in the front of the mouth, acrylic covering the biting surface of the back teeth 6, acrylic on the inside of the mouth on the side of the palate next to the molars 7, clasps 8 on the back teeth, and screws 13 placed into the acrylic at 20 degree vertical and ridge angles. The clasped appliance is for ages 7-adult.

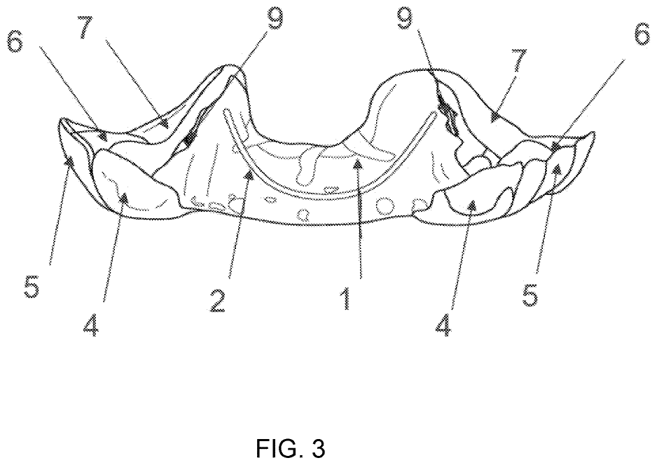

FIG. 3 is a front view of a claspless appliance oriented the same way it sits before it is placed into the mouth and over the teeth. Shown is the part of the acrylic that will be sitting against the palate and gum tissue with both sides showing the acrylic that will go over the teeth and be next to the inside of the cheeks. The appliance includes front acrylic section 1 against the front palate, gum tissue and the back side of the front teeth, support wire 2 on the inside of the acrylic in the front section of acrylic, distal on canines acrylic 4 over the distal, facial, and lingual of the upper canines and connected to the acrylic in the front of the mouth, acrylic on the outside of the baby molar teeth 5, acrylic covering the biting surface of the back teeth 6, acrylic on the inside of the mouth on the side of the palate next to the molars 7, and screws 9 placed into the acrylic at 3 degree vertical and widening angles. The claspless appliance is only for children ages 2-6.

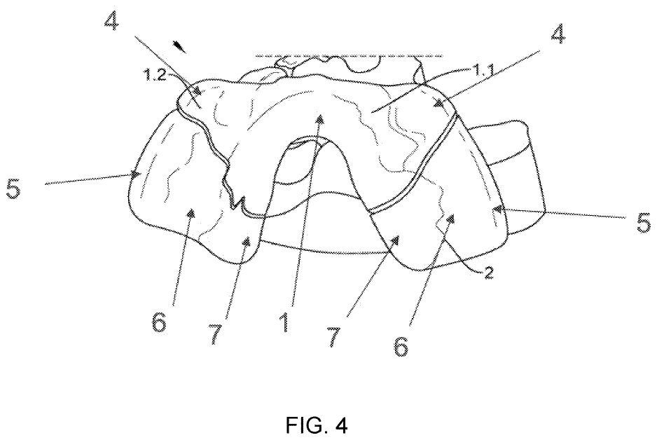

FIG. 4 is a bottom view of a claspless appliance leaning up against the model of a mouth. The front section of the appliance will be behind the front teeth. The smooth bottom part of the appliance is visible. The appliance includes front acrylic section 1 against the front palate, gum tissue and the back side of the front teeth, distal on canines acrylic 4 over the distal, facial, and lingual of the upper canines and connected to the acrylic in the front of the mouth, acrylic on the outside of the baby molar teeth 5, acrylic covering the biting surface of the back teeth 6, and acrylic on the inside of the mouth on the side of the palate next to the molars 7. The claspless appliance is only for children ages 2-6.

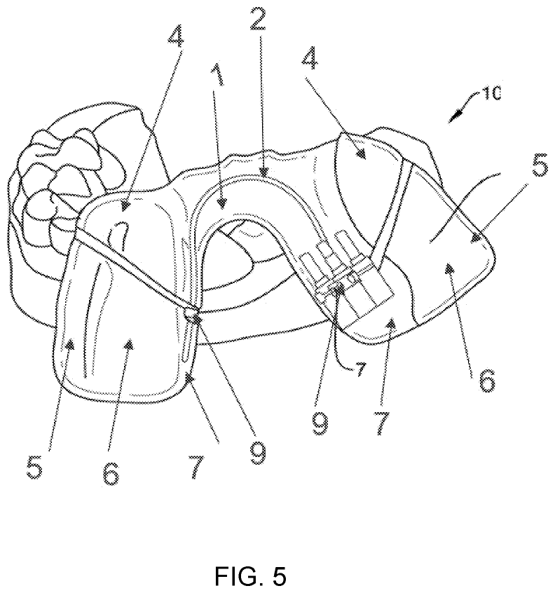

FIG. 5 is a bottom view of a claspless appliance 100 leaning up against the model of a mouth. The front section of the appliance will be behind the front teeth. The smooth bottom part of the appliance is visible. The appliance includes front acrylic section 1 against the front palate, gum tissue and the back side of the front teeth, support wire 2 on the inside of the acrylic in the front section of acrylic, distal on canines acrylic 4 over the distal, facial, and lingual of the upper canines and connected to the acrylic in the front of the mouth, acrylic on the outside of the baby molar teeth 5, acrylic covering the biting surface of the back teeth 6, acrylic on the inside of the mouth on the side of the palate next to the molars 7, and screws 9 placed into the acrylic at 3 degree vertical and widening angle. The claspless appliance is only for children ages 2-6.

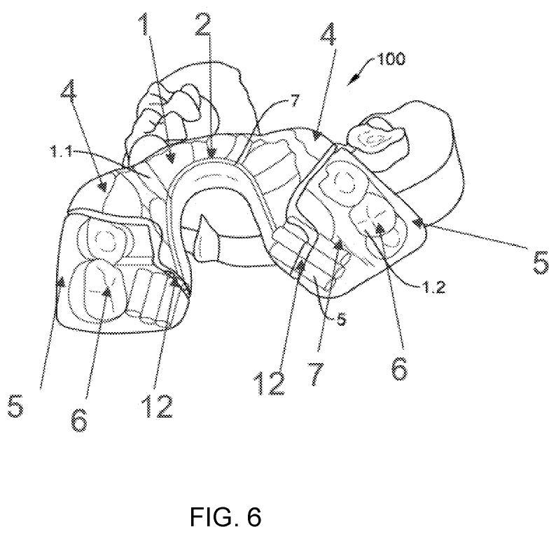

FIG. 6 is a bottom view of a claspless appliance 100 leaning up against the model of a mouth. The front section of the appliance will be behind the front teeth. The smooth bottom part of the appliance is visible. The appliance includes front acrylic section 1 against the front palate, gum tissue and the back side of the front teeth, support wire 2 on the inside of the acrylic in the front section of acrylic, distal on canines acrylic 4 over the distal, facial, and lingual of the upper canines and connected to the acrylic in the front of the mouth, acrylic on the outside of the baby molar teeth 5, acrylic covering the biting surface of the back teeth 6, acrylic on the inside of the mouth on the side of the palate next to the molars 7, and screws 12 placed into the acrylic at 15 degree vertical and widening angles. The claspless appliance is only for children ages 2-6.

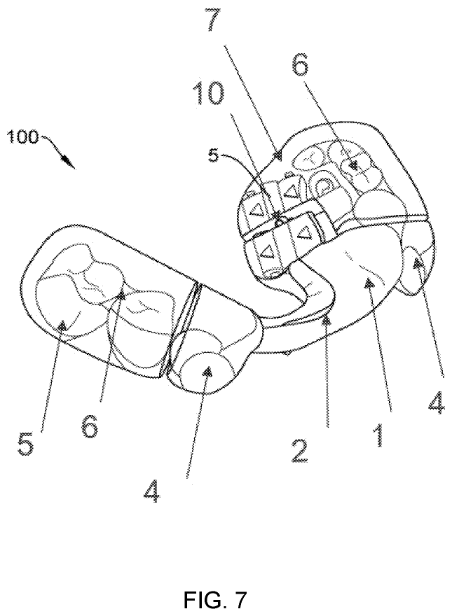

FIG. 7 is a bottom view of a claspless appliance 100 showing the biting surface and the front part of the appliance is toward the bottom right of the drawing. The appliance includes front acrylic section 1 against the front palate, gum tissue and the back side of the front teeth, support wire 2 on the inside of the acrylic in the front section of acrylic, distal on canines acrylic 4 over the distal, facial, and lingual of the upper canines and connected to the acrylic in the front of the mouth, acrylic on the outside of the baby molar teeth 5, acrylic covering the biting surface of the back teeth 6, acrylic on the inside of the mouth on the side of the palate next to the molars 7, and screws 10 placed into the acrylic at 5 degree vertical and ridge angles. The claspless appliance is only for children ages 2-6.

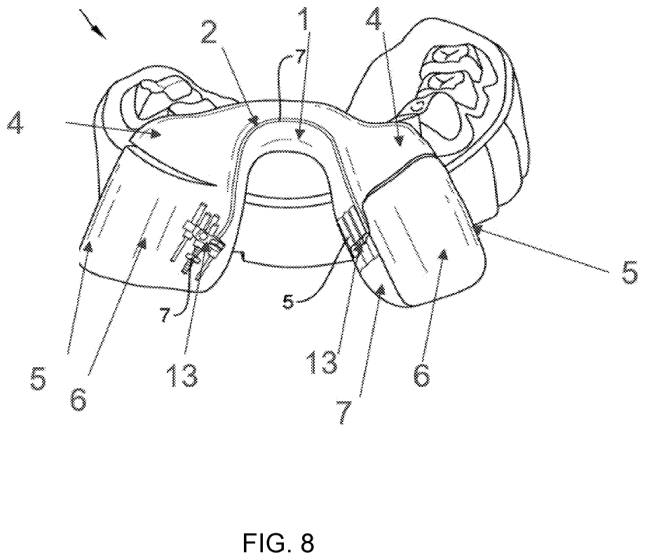

FIG. 8 is a bottom view of a claspless appliance leaning up against the model of a mouth. The front section of the appliance will be behind the front teeth. The smooth bottom part of the appliance is visible. The appliance includes front acrylic section 1 against the front palate, gum tissue and the back side of the front teeth, support wire 2 on the inside of the acrylic in the front section of acrylic, distal on canines acrylic 4 over the distal, facial, and lingual of the upper canines and connected to the acrylic in the front of the mouth, acrylic on the outside of the baby molar teeth 5, acrylic covering the biting surface of the back teeth 6, acrylic on the inside of the mouth on the side of the palate next to the molars 7, and screws 13 placed into the acrylic at 20 degree vertical and ridge angles. The claspless appliance is only for children ages 2-6.

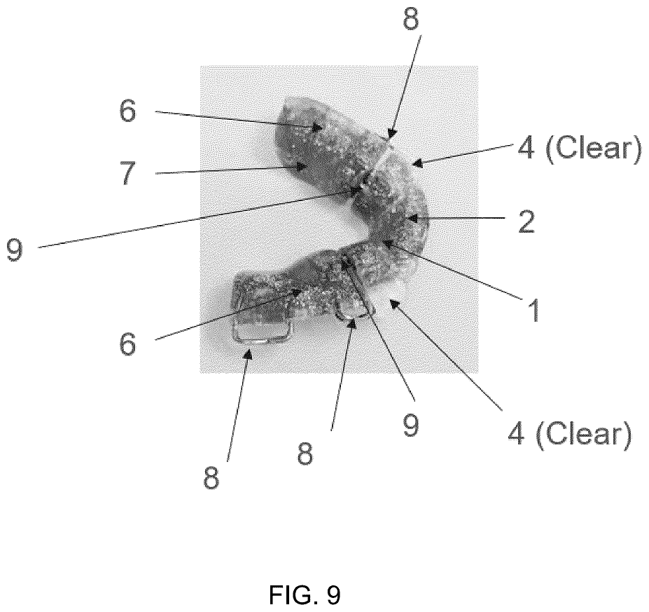

FIG. 9 is a plan view of a clasped appliance that is showing the part of the appliance that is visible when it is placed in a person's mouth. The front part of the appliance is toward the right of the drawing. The underside aspect is what would go against the palate, teeth and gums. The top part of the appliance is visible when it is placed in a person's mouth. The appliance includes front acrylic section 1 against the front palate, gum tissue and the back side of the front teeth, support wire 2 on the inside of the acrylic in the front section of acrylic, distal on canines acrylic 4 over the distal, facial, and lingual of the upper canines and connected to the acrylic in the front of the mouth, acrylic covering the biting surface of the back teeth 6, acrylic on the inside of the mouth on the side of the palate next to the molars 7, clasp 8 on the back teeth, and screws 9 placed into the acrylic at 3 degree vertical and widening angles. The clasped appliance is for ages 7-adult.

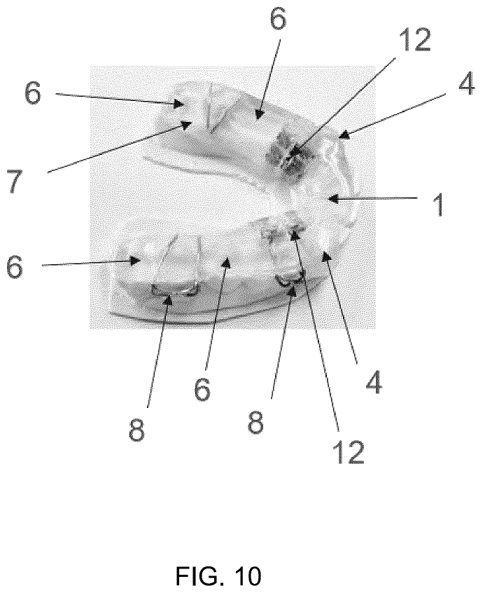

FIG. 10 is a plan view of a clasped appliance on a model of a mouth. The front of the mouth and appliance is toward the right side of the drawing. The appliance includes front acrylic section 1 against the front palate, gum tissue and the back side of the front teeth, distal on canines acrylic 4 over the distal, facial, and lingual of the upper canines and connected to the acrylic in the front of the mouth, acrylic covering the biting surface of the back teeth 6, acrylic on the inside of the mouth on the side of the palate next to the molars 7, clasp 8 on the back teeth, and screws 12 placed into the acrylic at 15 degree vertical and widening angles. The clasped appliance is for ages 7-adult.

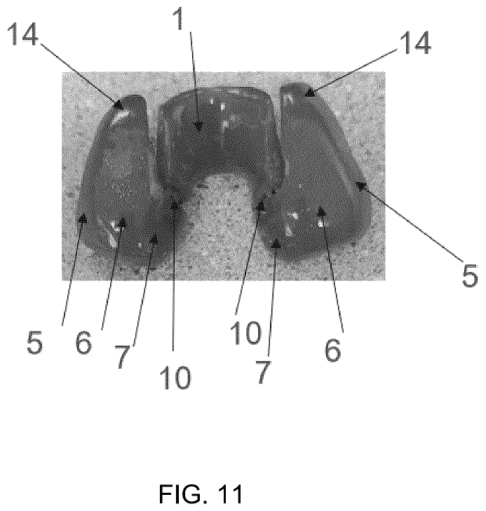

FIG. 11 is a plan view of a front four claspless appliance for only children ages 2-6. The visible aspect is what is visible when the appliance is in the mouth. The top part of the appliance in the drawing corresponds to the front of the mouth. The center section is what will be near the front of the child's mouth. The right and left side portions of the appliance fit over the baby canines and first baby molars. This appliance design is most often used when the second baby molars are not yet erupted. The appliance includes front acrylic section 1 against the front palate, gum tissue and the back side of the front teeth, acrylic on the outside of the baby molar teeth 5, acrylic covering the biting surface of the back teeth 6, acrylic on the inside of the mouth on the side of the palate next to the molars 7, screws 10 placed into the acrylic at 5 degree vertical and ridge angles, and acrylic completely over canines 14 connected to the acrylic in the back of the mouth.

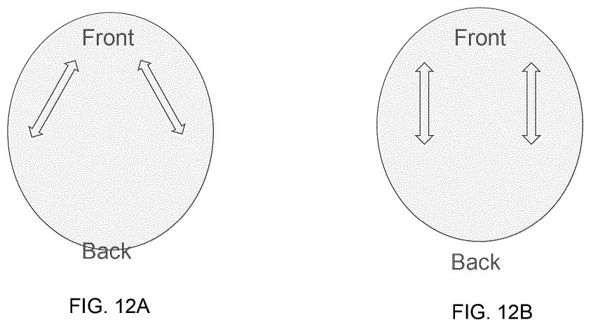

FIGS. 12A and 12B are schematic top views of an appliance demonstrating lateral angulation/side to side/width. In FIG. 12A the screws are placed at an acute angle to each other resulting in increased width near the bottom of the diagram or back of the mouth while the front of the upper jaw develops forward. The design of the front being lingual or distal on canines determines if the front teeth increase in circumference to unravel crowding or stay the same during this process. In FIG. 12B the screws are parallel resulting in forward development and no increase in width or no increased distance between the two sides at the bottom of the diagram or the back of the mouth.

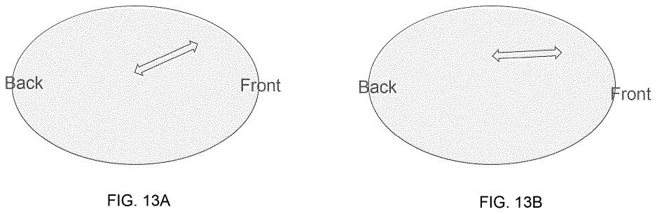

FIGS. 13A and 13B are schematic side views of an appliance demonstrating vertical angulation/up down. In FIG. 13A the screws are angling or pointing upward toward the front of the face or mouth. This will move the upper front teeth in a forward and upward direction resulting in someone showing a gummy smile to be less gummy. It will move the upper front teeth closer to the nose shortening that distance. FIG. 13B the screws are less angles and shows the forward or sagittal direction of movement without much vertical or upward component. There will still be some because of the shape of the palate, we are not putting pressure on a straight wall, the palate and teeth are sloped so that slope will also influence the direction of development.

The third plane of development is sagittal or front to back. All movements with the appliance can do forward or sagittal movements and development.

The present invention provides a removable airway appliance (100) for developing jaw structure. The screws are set at an up/down angle and a side-side angle. The screws are set on the appliance, and it is for the primary dentition on the baby teeth, a second system for permanent teeth, and for those that have some baby teeth and some permanent teeth.

In one embodiment, the present invention may grow the upper jaw forward and unrestrict the lower jaw that is often trapped back behind the upper jaw. The present invention guides and develops the lower and upper jaws forward, and widens the upper jaw, making room for the tongue in the mouth to maximize the airway space. The present removable airway appliance (100) guides and makes room for all the teeth, making room for all permanent teeth without crowding. Parents who begin their young children with this treatment can create space for all the permanent teeth before they come in and prevent crowding. The present invention develops the upper and lower jaws forward to get the back of the tongue forward away from the back of the throat which improves airflow for breathing and sleeping. The present invention helps in growth of the upper jaw forward and unrestrict the lower jaw that is often trapped back behind the upper jaw. The present invention provides better airway, better nasal breathing, upper and lower jaw forward growth, provides better cheek structure and better lip support to the human face. The removable airway appliance (100) can be worn 24 hours a day only removing a few minutes a day to brush for younger children (age 1-13) and worn 22 hours a day for older teenagers and adults age 14+. Removable airway appliance (100) method uses the removable airway appliance (100) in all primary dentition (all baby teeth) and permanent teeth for people from age 2 to older adult and it can be used with only 16 baby teeth.

The present invention reverses excess vertical/downward growth (controlling the amount and direction of growth/development) by using the removable airway appliance. The present invention controls growing jaws to improve airway (covering canines) or making room for teeth (acrylic behind canines) and provides a smooth surface over the biting area of the teeth and help to stimulate the forward growth of the lower jaw. The present invention creates a more open airway to breathe better and to reduce or eliminate sleep apnea.

The present invention uses in all primary dentition (all baby teeth) and permanent teeth for people from age 2 to older adult. The present invention is used with only 16 baby teeth and to reverse excess vertical/downward growth (controlling the amount and direction of growth/development). The present invention controls growing jaws to improve airway (covering canines) or making room for teeth (acrylic behind canines) and smooth surface over the biting area of the teeth help to stimulate the forward growth of the lower jaw.

The present invention creates a more open airway to breathe better and to reduce or eliminate sleep apnea. For wearing the appliance, the occlusal thickness of the expander is assessed using calipers and the lowest cusp on either side is measured from left to right to ensure proper thickness. Any type of adjustments are carried out if needed. The expander is applied in patient's mouth. Some precaution are taken before application of the appliance (100) like: acrylic is fully pressed up against the palate, the bite is checked to see if articulating paper holds on both sides for proper thickness. Care should be taken that the appliance (100) is not rocking in the mouth when they bite down, when patient is hitting both sides evenly, when they bite down. Care should be taken that the patient is not feeling any pain. If any adjustments are needed, are done to acrylic for removal of sharp or rough edges and care should be taken that an even occlusal surface is there from left to right. Instructions are given to the guardian/adult person on how to turn the key hold expander with the section that is up against the roof of the mouth towards the floor. The expander is hold securely where the key is being inserted. This prevents pressure that could break the expander. The key is inserted into the hole and the key is turned in the direction of the arrow, towards the floor. Turning the key to a full 90. It is always a “downhill” direction to expand. Reset the expander back to the beginning and allow the guardian to turn the key to ensure they understand watching for which direction, which keyhole and that they are supporting the expander well.

The steps for applying the removable airway appliance (100) of the present invention are as follows: Up-down angle reversing excess vertical/downward growth. This gets upward and forward development. This vertical angle can be 0 degrees, 5 degrees, 10 degrees, 15 degrees, 20 degrees, 25 degrees, 30 degrees.

Up-down angle parallel to teeth for upward and forward development.

Passive retention for all baby teeth and clasping system when permanent teeth come in.

Side angle used to control the amount of lateral/sideways growth and development.

Acrylic covering palate and back of front teeth to help control forces of forward/upward growth and development.

With an edge to edge or open bite, there is no overbite, the upper teeth have not come too far downward. There is not a gummy smile. However, the jaws still need to be developed forward. Developing the upper and lower jaws forward will give an improved airway for improved breathing and sleeping. The screws are positioned at a zero degree angle the direction of growth and development is in the forward direction. Again, this angle can be zero degrees to thirty degrees, using a higher degree angle when we want to correct a severe overbite, an excess of vertical growth or a gummy smile.

In another embodiment, the present invention has the retention through the complete coverage of the back teeth/back molars, for all primary/all baby teeth. There are no hooks or metal clasps hooking to the teeth to hold it in place. When permanent teeth are in then the retention is from the metal “hang clasps” that hook onto two of the back teeth on each side. The two metal hooks shown are the clasps that are on two back teeth on each side of the upper teeth. Another aspect is the amount of acrylic covering the palate/roof of the mouth and the lingual side/back side of the upper front teeth. When we want to change the vertical or up/down position of the front of the upper jaw we have less acrylic on the palate and more covering the back side of the upper teeth. When we want forward growth at zero or 5 degrees, we have the acrylic wider on the front palate/roof of the mouth and no acrylic on the back side of the upper front teeth. Anterior acrylic section covers a specified amount of the palate/roof of the mouth and lingual side/inside of top front teeth to direct the forces of development.

In combination, these unique features can combine in different ways for different applications such as for; severe overbite, moderate overbite, slight overbite, open bite, Class I, II, or III with Maxillary deficiency, and narrow or wide jaws. When the side-to-side position of the screws are set, they can be placed parallel for no lateral or no sideways development and only forward development. The side-to-side angle/position of the screws can be placed at as light angle to get increased width of the molars and a wider mouth as the forward growth is developed. The side-to-side angle of the screws can be put at a much steeper angle in relation to each other. This would be used for a very narrow mouth and achieve significant increased width or widening and lateral or sideways development of the mouth.

In case, when the mouth is very wide already and for forward development, the screws are placed parallel. When the mouth is average width, the screws are placed at a slight angle for gradual increase in width. When the mouth is very narrow, the screws are placed at an angle to increase the width faster.

In another embodiment, the removable airway appliance (100) is “U” shaped appliance having two protruded portions on the front side. The removable airway appliance (100) comprising of:
a. Anterior/Front acrylic section having a specified amount of the palate/roof of the mouth and lingual side/inside of top front teeth to direct the forces of development and a support wire embedded in the acrylic for added strength.
b. Posterior/back acrylic section providing retention over the primary/baby molars.
c. Expansion screws set at different angles for up/down and side/side growth and development having full coverage over canines.
d. Hang clasps or Richardson clasps for retention (in case if permanent teeth are present) including front clasp and back clasp.
e. Front support wire screw at 30°.

In another embodiment, in removable airway appliance, on the horizontal axis the screws can be positioned flat at a small angle or at a steeper angle to help direct the direction of growth and development of the jawbone. The side angle is directing the amount of increased width of the arch. Appliances are worn 24 hours a day only removing a few minutes a day to brush for younger children (age 1-13) and worn 22 hours a day for older teenagers and adults age 14+.

The screw assembly is fabricated of acrylic alloys. The front and back section are fabricated from acrylic. There is a complete acrylic coverage over the primary molars.

Both sides are connected by front support wire which maintains the desired lateral separation between front section and back section.

In another embodiment, 1. Stage 1 Early Primary Dentition Open Bite (in which Front teeth do not touch), Narrow Mouth Embodiment: 16 primary teeth, complete acrylic coverage over the primary first molar and primary canine. Acrylic behind the upper front teeth, screws parallel to teeth and ridge, screws at a 0 degree vertical angle (straight).

Stage 1 Early Primary Dentition Slight Overbite (1-30% Overbite), Narrow Mouth Embodiment: 16 primary teeth, complete acrylic coverage over the primary first molar and primary canine. Acrylic behind the upper front teeth, screws parallel to teeth and ridge, screws at a 10 degree angle upward.

Stage 1 Early Primary Dentition Moderate Overbite (31-65% Overbite), Narrow Mouth Embodiment: 16 primary teeth, complete acrylic coverage over the primary first molar and primary canine. Acrylic behind the upper front teeth, screws parallel to teeth and ridge, screws at a 20 degree angle upward.

Stage 1 Early Primary Dentition Deep Bite (66-100% Overbite), Narrow Mouth Embodiment: 16 primary teeth, complete acrylic coverage over the primary first molar and primary canine. Acrylic behind the upper front teeth, screws parallel to teeth and ridge, screws at a 30 degree angle upward.

Stage 1 Early Primary Dentition Open Bite (Front teeth do not touch), Wide Mouth Embodiment: 16 primary teeth, complete acrylic coverage over the primary first molar and primary canine. Acrylic behind the upper front teeth, screws parallel to each other, screws at a 30 degree angle upward.

Stage 1 Early Primary Dentition Slight Overbite (1-30% Overbite), Wide Mouth Embodiment: 16 primary teeth, complete acrylic coverage over the primary first molar and primary canine. Acrylic behind the upper front teeth, screws parallel to each other, screws at a 30 degree angle upward.

Stage 1 Early Primary Dentition Moderate Overbite (31-65% Overbite), Wide Mouth Embodiment: 16 primary teeth, complete acrylic coverage over the primary first molar and primary canine. Acrylic behind the upper front teeth, screws parallel to each other, screws at a 30 degree angle upward.

Stage 1 Early Primary Dentition Deep Bite (66-100% Overbite), Wide Mouth Embodiment: 16 primary teeth, complete acrylic coverage over the primary first molar and primary canine. Acrylic behind the upper front teeth, screws parallel to each other, screws at a 30 degree angle upward.

Stage 1 Primary Dentition Open Bite (Front teeth do not touch), Narrow Mouth Embodiment: 20 primary teeth, complete acrylic coverage over the primary molars. Acrylic behind the upper front teeth, screws parallel to teeth and ridge, screws at a 0 degree vertical angle (straight).

Stage 1 Primary Dentition Slight Overbite (1-30% Overbite), Narrow Mouth Embodiment: 20 primary teeth, complete acrylic coverage over the primary molars. Acrylic behind the upper front teeth, screws parallel to teeth and ridge, screws at a 10 degree angle upward.

Stage 1 Primary Dentition Moderate Overbite (31-65% Overbite), Narrow Mouth Embodiment: 20 primary teeth, complete acrylic coverage over the primary molars. Acrylic behind the upper front teeth, screws parallel to teeth and ridge, screws at a 20 degree angle upward.

Stage 1 Primary Dentition Deep Bite (66-100% Overbite), Narrow Mouth Embodiment: 20 primary teeth, complete acrylic coverage over the primary molars. Acrylic behind the upper front teeth, screws parallel to teeth and ridge, screws at a 30 degree angle upward.

Stage 1 Primary Dentition Open Bite (Front teeth do not touch), Wide Mouth Embodiment: 20 primary teeth, complete acrylic coverage over the primary molars. Acrylic behind the upper front teeth, screws parallel to each other, screws at a 30 degree angle upward.

Stage 1 Primary Dentition Slight Overbite (1-30% Overbite), Wide Mouth Embodiment: 20 primary teeth, complete acrylic coverage over the primary molars. Acrylic behind the upper front teeth, screws parallel to each other, screws at a 30 degree angle upward.

Stage 1 Primary Dentition Moderate Overbite (31-65% Overbite), Wide Mouth Embodiment: 20 primary teeth, complete acrylic coverage over the primary molars. Acrylic behind the upper front teeth, screws parallel to each other, screws at a 30 degree angle upward.

Stage 1 Primary Dentition Deep Bite (66-100% Overbite), Wide Mouth Embodiment: 20 primary teeth, complete acrylic coverage over the primary molars. Acrylic behind the upper front teeth, screws parallel to each other, screws at a 30 degree angle upward.

Stage 1 Mixed Dentition Open Bite (Front teeth do not touch), Narrow Mouth Embodiment: 20 primary teeth, complete acrylic coverage over the primary molars. Acrylic behind the upper front teeth, screws parallel to teeth and ridge, screws at a 0 degree vertical angle (straight).

Stage 1 Mixed Dentition Slight Overbite (1-30% Overbite), Narrow Mouth Embodiment: 20 primary teeth, complete acrylic coverage over the primary molars. Acrylic behind the upper front teeth, screws parallel to teeth and ridge, screws at a 10 degree angle upward.

Stage 1 Mixed Dentition Moderate Overbite (31-65% Overbite), Narrow Mouth Embodiment: 20 primary teeth, complete acrylic coverage over the primary molars. Acrylic behind the upper front teeth, screws parallel to teeth and ridge, screws at a 20 degree angle upward.

Stage 1 Mixed Dentition Deep Bite (66-100% Overbite), Narrow Mouth Embodiment: 20 primary teeth, complete acrylic coverage over the primary molars. Acrylic behind the upper front teeth, screws parallel to teeth and ridge, screws at a 30 degree angle upward.

Stage 1 Mixed Dentition Open Bite (Front teeth do not touch), Wide Mouth Embodiment: 20 primary teeth, complete acrylic coverage over the primary molars. Acrylic behind the upper front teeth, screws parallel to each other, screws at a 30 degree angle upward.

Stage 1 Mixed Dentition Slight Overbite (1-30% Overbite), Wide Mouth Embodiment: 20 primary teeth, complete acrylic coverage over the primary molars. Acrylic behind the upper front teeth, screws parallel to each other, screws at a 30 degree angle upward.

Stage 1 Mixed Dentition Moderate Overbite (31-65% Overbite), Wide Mouth Embodiment: 20 primary teeth, complete acrylic coverage over the primary molars. Acrylic behind the upper front teeth, screws parallel to each other, screws at a 30 degree angle upward.

Stage 1 Mixed Dentition Deep Bite (66-100% Overbite), Wide Mouth Embodiment: 20 primary teeth, complete acrylic coverage over the primary molars. Acrylic behind the upper front teeth, screws parallel to each other, screws at a 30 degree angle upward.

Stage 1 Permanent Dentition Open Bite (Front teeth do not touch), Narrow Mouth Embodiment: 20 primary teeth, complete acrylic coverage over the primary molars. Acrylic behind the upper front teeth, screws parallel to teeth and ridge, screws at a 0 degree vertical angle (straight).

Stage 1 Permanent Dentition Slight Overbite (1-30% Overbite), Narrow Mouth Embodiment: 20 primary teeth, complete acrylic coverage over the primary molars. Acrylic behind the upper front teeth, screws parallel to teeth and ridge, screws at a 10 degree angle upward.

Stage 1 Permanent Dentition Moderate Overbite (31-65% Overbite), Narrow Mouth Embodiment: 20 primary teeth, complete acrylic coverage over the primary molars. Acrylic behind the upper front teeth, screws parallel to teeth and ridge, screws at a 20 degree angle upward.

Stage 1 Permanent Dentition Deep Bite (66-100% Overbite), Narrow Mouth Embodiment: 20 primary teeth, complete acrylic coverage over the primary molars. Acrylic behind the upper front teeth, screws parallel to teeth and ridge, screws at a 30 degree angle upward.

Stage 1 Permanent Dentition Open Bite (Front teeth do not touch), Wide Mouth Embodiment: 20 primary teeth, complete acrylic coverage over the primary molars. Acrylic behind the upper front teeth, screws parallel to each other, screws at a 30 degree angle upward.

Stage 1 Permanent Dentition Slight Overbite (1-30% Overbite), Wide Mouth Embodiment: 20 primary teeth, complete acrylic coverage over the primary molars. Acrylic behind the upper front teeth, screws parallel to each other, screws at a 30 degree angle upward.

Stage 1 Permanent Dentition Moderate Overbite (31-65% Overbite), Wide Mouth Embodiment: 20 primary teeth, complete acrylic coverage over the primary molars. Acrylic behind the upper front teeth, screws parallel to each other, screws at a 30 degree angle upward.

Stage 1 Permanent Dentition Deep Bite (66-100% Overbite), Wide Mouth Embodiment: 20 primary teeth, complete acrylic coverage over the primary molars. Acrylic behind the upper front teeth, screws parallel to each other, screws at a 30 degree angle upward.

In another embodiment,

Stage 2 Primary Dentition Open Bite (Front teeth do not touch), Narrow Mouth Embodiment: 20 primary teeth, complete acrylic coverage over the primary molars. Acrylic completely over the upper canines and behind the other upper front teeth, screws parallel to teeth and ridge, screws at a 0 degree vertical angle (straight).

Stage 2 Primary Dentition Slight Overbite (1-30% Overbite), Narrow Mouth Embodiment: 20 primary teeth, complete acrylic coverage over the primary molars. Acrylic completely over the upper canines and behind the other upper front teeth, screws parallel to teeth and ridge, screws at a 10 degree angle upward.

Stage 2 Primary Dentition Moderate Overbite (31-65% Overbite), Narrow Mouth Embodiment: 20 primary teeth, complete acrylic coverage over the primary molars. Acrylic completely over the upper canines and behind the other upper front teeth, screws parallel to teeth and ridge, screws at a 20 degree angle upward.

Stage 2 Primary Dentition Deep Bite (66-100% Overbite), Narrow Mouth Embodiment: 20 primary teeth, complete acrylic coverage over the primary molars. Acrylic completely over the upper canines and behind the other upper front teeth, screws parallel to teeth and ridge, screws at a 30 degree angle upward.

Stage 2 Primary Dentition Open Bite (Front teeth do not touch), Wide Mouth Embodiment: 20 primary teeth, complete acrylic coverage over the primary molars. Acrylic completely over the upper canines and behind the other upper front teeth, screws parallel to each other, screws at a 30 degree angle upward.

Stage 2 Primary Dentition Slight Overbite (1-30% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.

Stage 2 Primary Dentition Moderate Overbite (31-65% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.

Stage 2 Primary Dentition Deep Bite (66-100% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.

Stage 2 Mixed Dentition Open Bite (Front teeth do not touch), Narrow Mouth Embodiment: 20 primary teeth, complete acrylic coverage over the primary molars. Acrylic completely over the upper canines and behind the other upper front teeth, screws parallel to teeth and ridge, screws at a 0 degree vertical angle (straight).

Stage 2 Mixed Dentition Slight Overbite (1-30% Overbite), Narrow Mouth Embodiment: 20 primary teeth, complete acrylic coverage over the primary molars. Acrylic completely over the upper canines and behind the other upper front teeth, screws parallel to teeth and ridge, screws at a 10 degree angle upward.

Stage 2 Mixed Dentition Moderate Overbite (31-65% Overbite), Narrow Mouth Embodiment: 20 primary teeth, complete acrylic coverage over the primary molars. Acrylic completely over the upper canines and behind the other upper front teeth, screws parallel to teeth and ridge, screws at a 20 degree angle upward.

Stage 2 Mixed Dentition Deep Bite (66-100% Overbite), Narrow Mouth Embodiment: 20 primary teeth, complete acrylic coverage over the primary molars. Acrylic completely over the upper canines and behind the other upper front teeth, screws parallel to teeth and ridge, screws at a 30 degree angle upward.

Stage 2 Mixed Dentition Open Bite (Front teeth do not touch), Wide Mouth Embodiment: 20 primary teeth, complete acrylic coverage over the primary molars. Acrylic completely over the upper canines and behind the other upper front teeth, screws parallel to each other, screws at a 30 degree angle upward.

Stage 2 Mixed Dentition Slight Overbite (1-30% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.

Stage 2 Mixed Dentition Moderate Overbite (31-65% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.

Stage 2 Mixed Dentition Deep Bite (66-100% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.

Stage 2 Permanent Dentition Open Bite (Front teeth do not touch), Narrow Mouth Embodiment: 20 primary teeth, complete acrylic coverage over the primary molars. Acrylic completely over the upper canines and behind the other upper front teeth, screws parallel to teeth and ridge, screws at a 0 degree vertical angle (straight).

Stage 2 Permanent Dentition Slight Overbite (1-30% Overbite), Narrow Mouth Embodiment: 20 primary teeth, complete acrylic coverage over the primary molars. Acrylic completely over the upper canines and behind the other upper front teeth, screws parallel to teeth and ridge, screws at a 10 degree angle upward.

Stage 2 Permanent Dentition Moderate Overbite (31-65% Overbite), Narrow Mouth Embodiment: 20 primary teeth, complete acrylic coverage over the primary molars. Acrylic completely over the upper canines and behind the other upper front teeth, screws parallel to teeth and ridge, screws at a 20 degree angle upward.

Stage 2 Permanent Dentition Deep Bite (66-100% Overbite), Narrow Mouth Embodiment: 20 primary teeth, complete acrylic coverage over the primary molars. Acrylic completely over the upper canines and behind the other upper front teeth, screws parallel to teeth and ridge, screws at a 30 degree angle upward.

Stage 2 Permanent Dentition Open Bite (Front teeth do not touch), Wide Mouth Embodiment: 20 primary teeth, complete acrylic coverage over the primary molars. Acrylic completely over the upper canines and behind the other upper front teeth, screws parallel to each other, screws at a 30 degree angle upward.

Stage 2 Permanent Dentition Slight Overbite (1-30% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.

Stage 2 Permanent Dentition Moderate Overbite (31-65% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.

Stage 2 Permanent Dentition Deep Bite (66-100% Overbite), Wide Mouth Embodiment: 20 primary teeth, complete acrylic coverage over the primary molars. Acrylic completely over the upper canines and behind the other upper front teeth, screws parallel to each other, screws at a 30 degree angle upward.

The removable appliance (100) can be taken out, the patient comes in for simple adjustments every four to twelve weeks. The upper arch expands anteriorly (to the front) by approximately 1 mm a month. Once the desired spaces are achieved between the upper canines and the first primary molars behind, the appliance (100) is removed and a retainer is inserted inside the mouth to avoid relapse, to make sure that the teeth do not collapse back to the original position. After the permanent teeth come in the bone is stable. Braces may not be needed in all cases. If needed, braces are placed on both upper and lower teeth. The braces with the help of elastic bands moves the back teeth forward to close the gaps, while maintaining the position of the front teeth. This procedure enhances the chance of all wisdom teeth erupting in the correct position, without the need for extractions or headgear.

In the meantime, the lower braces can straighten the lower teeth if needed. Once all the gaps are closed, the upper and lower teeth are coordinated to ensure optimal aesthetics and function.

The end result is an expanded upper jaw both towards the front and the sides. The jaw is able to accommodate all permanent teeth (may include wisdom teeth if the conditions are right) to erupt in the correct position, the lower jaw is able to occlude into the ideal bite in harmony with the TMJ joints and the facial muscles. The ensuing enlarged space in the mouth allows the tongue to position itself correctly against the roof of the mouth, opening good airways at the back of the throat and inside the nose. The removable appliance (100) is designed for children to accommodate the orofacial growth. The appliance (100) is turned (activated) twice a week by the patients themselves, or for the very young ones, by their parents. It is to be worn 24/7 including eating. The appliance (100) expands the upper jaw in all dimensions, to lay down the foundation and to accommodate the eruption of the incoming permanent teeth. When the permanent teeth are ready to erupt down there will be adequate amount of space.

The working of the removable airway appliance (100) is as follows.

The removable airway appliance (100) is attached to back teeth and gently, pressure is put on the nasopalatine nerve that runs through palate as well as right forward pressure on the bone and the canines and front teeth stimulating bone remodeling and development. This pressure stimulates forward development of the upper jaw, which also allows the lower jaw to develop forward. When this development takes place, it can create room for even severely crowded teeth to align, and it can open up the airway, enlarge your airway and move the mid and lower face forward. The present invention helps to expand the jaws forward without the need for surgery. More benefits can include long-term relief from issues like TMD pain and Sleep Apnea without the long-term use of oral appliances. The present invention helps in sleep apnea and snoring because when a person undergoes the maxillary expansion of jaw, the airway can be opened. And when upper jaw expands during this treatment process, it can effectively realign the lower jaw, helping to relieve TMD clicking, popping and pain without surgery. This happens because as the upper and lower jaws develop forward, there is more space for the disc of the TMJ to function smoothly.

Up-down angle parallel to teeth for upward and forward development. Up-down angle reverses excess vertical/downward growth. This gets upward and forward development. This angle can be 0 degrees, 5 degrees, 10 degrees, 15 degrees, 20 degrees, 25 degrees, 30 degrees. The side angle used to control the amount of lateral/sideways growth and development. Acrylic covering palate and back of front teeth to help control forces of forward/upward growth and development.

In some implementations, the airway appliance may include one or more of three different screw positions to control and direct bone development and shape of the jaws: (1) Down and back screw position with the screws placed higher in the palate and over the gums BELOW the 2nd premolar or 2nd baby molar when on the model and ABOVE the teeth higher in the palate when in the mouth; (2) Up and forward when increased widening is desired as well as minimal vertical changes in the upper jaw and bite. This screw position is placing the middle of the screw between the canine and first premolar or canine and first baby molar right next to the teeth; and (3) Up and back when minimal to no increased width is desired and more forward focused jaw development is desired. This screw position is placing the screw parallel to the posterior teeth or parallel to each other alongside the premolars and first molar. Minimal to no increase in width and mostly forward development with minimal changes in the bite and vertical position of the teeth.

In some implementations, the airway appliance may include one or more of a clasp design and style: (a) Hang type clasp with two separate wires coming out of the acrylic, crossing over each other next to the tooth and engaging into the interproximal surface of the tooth, and (b) Richardson style clasp with two separate swooping wires being soldered together with one wire arcing into the acrylic on both sides of the teeth and the other wire arcing into the interproximal areas of the teeth with a solder joint when the pass next to each other about mid tooth. The Hang style clasp has been used by Dr. Bill Hang or William Hang previously. The Richardson style clasp gives a lower profile clasp that protrudes out less and irritates the cheek less and is more esthetic.

In some implementations, the airway appliance may include one or more of an occlusal surface coverage: (a) 2 mm flat occlusal coverage all the way front to back. This will often have the bite being on the very back or distal surface of the appliance to promote a counterclockwise rotation of the Mandible; (b) 2 mm flat occlusal coverage in the front and middle with a 30 degree drop or decline on the back or distal tooth. This allows for the bite to be hitting in the middle of the appliance; (c) 1.5 mm to 2 mm incline occlusal coverage to allow for an even bite on the acrylic; and (d) 3 mm or 4 mm occlusal coverage when there is an anterior crossbite (lower front teeth in front of upper front teeth) and we need more vertical space to jump the crossbite without the upper and lower front teeth being damaged as they cross over each other.

In some implementations, the airway appliance may include one or more of Anterior acrylic design specifications: (a) Distal on the canines on primary canines means the primary canine is fully covered by acrylic on all surfaces to bodily move the canine and front teeth forward bodily with minimal tipping; (b) Distal on the canines on permanent canines means the acrylic will cover ¼ to ⅓ of the incisal edge or tip edge of the permanent canine to guide forward development with minimal flaring and mostly bodily movement as the upper jaw develops forward; and (c) Distal on the laterals when the permanent canines are blocked out and not erupted into proper position and space needs to be created for the permanent canine teeth. The acrylic covers about ¼ of the incisal edge of the lateral incisors to control the forward development and minimize tipping of the front teeth as the Maxilla develops forward.

Advantages of the present invention include:

The present invention has uses in all primary dentition (all baby teeth) and permanent teeth for people from age 2 to older adult and can be used not only in the all primary dentition, but also with only 16 baby teeth. The present invention has the ability to reverse excess vertical/downward growth (controlling the amount and direction of growth/development) and has the ability to control growing jaws to improve airway (covering canines) or making room for teeth (acrylic behind canines). Smooth surface over the biting area of the teeth help to stimulate the forward growth of the lower jaw and creates a more open airway to breathe better and to reduce or eliminate sleep apnea. The present invention helps to grow and develop the jaw structures permanently in more forward and ideal position. The present invention helps develops the bone of the upper and lower jaws forward permanently. It can non-surgically develop the Maxilla in an upward direction reversing a gummy smile.

Accordingly, the removable airway appliance may be characterized as a “U” shaped removable airway appliance having two protruded portions configured to reverse excess vertical/downward growth and develop the upper and lower jaw bones permanently in a forward position, the appliance having two sections including a front/anterior acrylic material section and a back/posterior acrylic material section held together with a set of two expansion screws that can be adjusted in a lateral or vertical direction for controlling or reversing the direction of growth while enhancing the palate and airway space, including one or more of: (a) the anterior/Front acrylic material section is capable of covering an amount of the palate/roof of the mouth and lingual side/inside of top front teeth development; having full coverage over canines, customizable to cover 0 mm, 2 mm, 4 mm, 6 mm, or 8 mm of the palate measured from the gumline behind the front teeth toward the throat, and 0 mm, 2 mm, 4 mm, or 6 mm on the back of the front teeth measured from the gumline toward the edge, with encapsulating canines for bodily forward movement or lingual adaptation for crowding in the front six teeth; (b) a support wire is embedded in the acrylic material for added strength, made of chrome/cobalt alloy in the front section for all retention styles and canine configurations; (c) the posterior/back acrylic material section providing retention over the primary/baby molars; (d) the expansion screws set at different angles for up/down and side/side growth and development, consisting of two screws capable of 0-30 degrees vertically to reverse downward growth and laterally for width control, enabling treatment impact over canines through the acrylic without direct screw coverage; (e) hang clasps for retention, including a front clasp and a back clasp made of chrome/cobalt alloy wire engaging four posterior teeth; and (f) a front support wire at 30 degrees being embedded chrome/cobalt alloy wire for strength.

The removable airway appliance may also be characterized as a “U” shaped removable airway appliance having two protruded portions on the front side, configured to reverse excessing vertical/downward growth and developing the upper and lower jaws bones permanently in a forward position, wherein the appliance is configured to use in all primary dentition (all baby teeth) and permanent teeth for people from age 1 (with 16 baby teeth) to older adults.

The removable airway appliance may also be characterized as a “U” shaped removable airway appliance having two protruded portions on the front side, configured to reverse excess vertical/downward growth and develop the upper and lower jaw bones permanently in a forward position, wherein a mean thickness of the appliance is assessed using calipers and the lowest cusp on either side is measured from left to right to ensure proper thickness, the mean thickness being the acrylic thickness over the biting surface or occlusal surface of the teeth.

The removable airway appliance may also be characterized as a “U” shaped removable airway appliance having two protruded portions on the front side, configured to reverse excess vertical/downward growth and develop the upper and lower jaws bones permanently in a forward position, wherein the appliance is configured to be applied in patient's mouth and held securely for insertion of a key into a hole and is configured to turning of the key in the direction of an arrow at an angle of 90 degrees.

The removable airway appliance may also be characterized as a “U” shaped removable airway appliance having two protruded portions on the front side, configured to reverse excess vertical/downward growth and developing the upper and lower jaws bones permanently in a forward position, wherein the expansion screws set is positioned at a zero degree to thirty degrees, using a higher degree angle if the direction of growth and development is in the forward direction.

The removable airway appliance may also be characterized as a “U” shaped removable airway appliance having two protruded portions on the front side, configured to reverse excess vertical/downward growth and developing the upper and lower jaws bones permanently in a forward position, wherein the appliance is configured to be combined in different ways for different applications such as for; severe overbite, moderate overbite, slight overbite, open bite, Class I, II, or III with Maxillary deficiency, and narrow or wide jaws.

The removable airway appliance may also be characterized as a “U” shaped removable airway appliance having two protruded portions on the front side, configured to reverse excess vertical/downward growth and developing the upper and lower jaws bones permanently in a forward position, wherein the appliance is configured to be worn 24 hours a day (only removing a few minutes a day to brush) for younger children (age 1-13) or worn 20-22 hours a day for older teenagers and adults age 14+.

The invention may also be characterized as a process for application of a removable airway appliance comprising the steps of (a) reversing an up-down angle at 0 degrees, 5 degrees, 10 degrees, 15 degrees, 20 degrees, 25 degrees, 30 degrees for excess vertical/downward growth; (b) resetting the up-down angle parallel to teeth for upward and forward development; (c) providing a passive retention means for all baby teeth or a clasping system for permanent teeth; (d) controlling the amount of lateral/sideways growth using side angle for development; and/or (e) providing an acrylic covering palate and back of front teeth for controlling forces of forward/upward growth and development.

The removable airway appliance may also be characterized as an appliance configured to guide development of a patient's maxillary and mandibular structures, comprising: a U-shaped appliance body sized to be removably positioned within an upper dental arch of a patient; an anterior acrylic section configured to contact at least a portion of a palate and a lingual surface of one or more anterior teeth; a posterior acrylic section configured to extend over posterior teeth of the upper dental arch; at least two expansion screws embedded within the appliance body and extending between portions of the appliance body on opposite sides of the dental arch; wherein the expansion screws are positioned at predetermined angular orientations relative to a horizontal plane of the dental arch so as to direct orthodontic development of the maxilla in a forward direction and optionally in a vertical and/or lateral direction; and wherein the anterior acrylic section and the posterior acrylic section cooperate to transmit forces generated by the expansion screws to promote forward development of the maxillary structure and increased airway space.

The appliance may also be characterized as the appliance described above wherein the expansion screws are oriented at a vertical angle between 0 degrees and 30 degrees relative to the horizontal plane of the dental arch.

The appliance may also be characterized as the appliance described above wherein the expansion screws are oriented relative to each other at a lateral angle configured to control lateral expansion of the dental arch.

The appliance may also be characterized as the appliance described above wherein the expansion screws are positioned parallel to each other to promote primarily sagittal forward development of the maxilla.

The appliance may also be characterized as the appliance described above wherein the expansion screws are positioned at an acute angle relative to each other to produce lateral widening of the dental arch during forward development.

The appliance may also be characterized as the appliance described above wherein the anterior acrylic section covers a portion of the palate and a lingual surface of the anterior teeth to direct orthodontic forces applied to the maxilla.

The appliance may also be characterized as the appliance described above wherein the posterior acrylic section provides retention by covering occlusal surfaces of posterior teeth.

The appliance may also be characterized as the appliance described above wherein the posterior acrylic section provides complete acrylic coverage over primary molars.

The appliance may also be characterized as the appliance described above further comprising at least one (e.g., one or two) support wire embedded within the anterior acrylic section to reinforce the appliance.

The appliance may also be characterized as the appliance described above wherein the one or two support wires comprise a chrome-cobalt alloy wire.

The appliance may also be characterized as the appliance described above further comprising one or more clasps configured to engage posterior teeth for retention.

The appliance may also be characterized as the appliance described above wherein the appliance includes occlusal acrylic coverage extending across biting surfaces of posterior teeth.

The appliance may also be characterized as the appliance described above wherein the occlusal acrylic coverage has a thickness between approximately 1.5 mm and 4 mm.

The appliance may also be characterized as the appliance described above wherein the appliance includes acrylic coverage around canine teeth configured to direct forward movement of anterior teeth during expansion.

The appliance may also be characterized as the appliance described above wherein the anterior acrylic section covers between 0 mm and 8 mm of the palate measured from a gumline behind the anterior teeth.

The appliance may also be characterized as the appliance described above wherein the appliance is configured for use in primary dentition, mixed dentition, or permanent dentition.

The appliance may also be characterized as an orthodontic development system, comprising: a removable airway appliance configured to be positioned in an upper dental arch of a patient; the appliance including: an anterior acrylic section configured to contact a palate and lingual surfaces of anterior teeth; a posterior acrylic section covering occlusal surfaces of posterior teeth; two expansion screws embedded within the appliance and extending between opposite sides of the appliance; wherein the expansion screws are adjustable and positioned at selected vertical and lateral angles to control a direction of maxillary development including forward sagittal development, lateral arch expansion, and vertical positional adjustment of anterior teeth; and whereby the appliance promotes forward development of the maxilla and enlargement of an airway space.

The invention may also be characterized as a method for guiding development of a maxilla and airway of a patient, comprising: positioning a removable orthodontic airway appliance within an upper dental arch of a patient, the appliance including an anterior acrylic section, a posterior acrylic section, and at least two expansion screws; orienting the expansion screws at selected vertical and lateral angles; periodically activating the expansion screws to apply orthodontic forces to the palate and teeth; and directing maxillary development in a forward direction while optionally increasing arch width and modifying vertical tooth position.

The invention may also be characterized as the method described above further comprising adjusting the expansion screws approximately twice per week.

The invention may also be characterized as the method described above wherein the appliance is used to increase airway space by repositioning the maxilla and allowing forward positioning of the mandible.

The above disclosure is sufficient to enable one of ordinary skill in the art to practice the invention, and provides the best mode of practicing the invention presently contemplated by the inventor. While there is provided herein a full and complete disclosure of the preferred embodiments of this invention, it is not desired to limit the invention to the exact construction, dimensional relationships, and operation shown and described. Various modifications, alternative constructions, changes and equivalents will readily occur to those skilled in the art and may be employed, as suitable, without departing from the true spirit and scope of the invention. Such changes might involve alternative materials, components, structural arrangements, sizes, shapes, forms, functions, operational features or the like.

Therefore, the above description and illustrations should not be construed as limiting the scope of the invention, which is defined by the appended claims.

What is claimed as invention is:

1. A removable orthodontic airway appliance configured to guide development of a patient's maxillary and mandibular structures, comprising:
a U-shaped appliance body sized to be removably positioned within an upper dental arch of a patient;
an anterior acrylic section configured to contact at least a portion of a palate and a lingual surface of one or more anterior teeth;
a posterior acrylic section configured to extend over posterior teeth of the upper dental arch;
at least two expansion screws embedded within the appliance body and extending between portions of the appliance body on opposite sides of the dental arch;
wherein the expansion screws are positioned at predetermined angular orientations relative to a horizontal plane of the dental arch so as to direct orthodontic development of the maxilla in a forward direction and optionally in a vertical and/or lateral direction; and
wherein the anterior acrylic section and the posterior acrylic section cooperate to transmit forces generated by the expansion screws to promote forward development of the maxillary structure and increased airway space.

2. The appliance of claim 1, wherein the expansion screws are oriented at a vertical angle between 0 degrees and 30 degrees relative to the horizontal plane of the dental arch.

3. The appliance of claim 1, wherein the expansion screws are oriented relative to each other at a lateral angle configured to control lateral expansion of the dental arch.

4. The appliance of claim 3, wherein the expansion screws are positioned parallel to each other to promote primarily sagittal forward development of the maxilla.

5. The appliance of claim 3, wherein the expansion screws are positioned at an acute angle relative to each other to produce lateral widening of the dental arch during forward development.

6. The appliance of claim 1, wherein the anterior acrylic section covers a portion of the palate and a lingual surface of the anterior teeth to direct orthodontic forces applied to the maxilla.

7. The appliance of claim 1, wherein the posterior acrylic section provides retention by covering occlusal surfaces of posterior teeth.

8. The appliance of claim 7, wherein the posterior acrylic section provides complete acrylic coverage over primary molars.

9. The appliance of claim 1, further comprising at least one support wire embedded within the anterior acrylic section to reinforce the appliance.

10. The appliance of claim 9, wherein the at least one support wire comprises a chrome-cobalt alloy wire.

11. The appliance of claim 1, further comprising one or more clasps configured to engage posterior teeth for retention.

12. The appliance of claim 1, wherein the appliance includes occlusal acrylic coverage extending across biting surfaces of posterior teeth.

13. The appliance of claim 12, wherein the occlusal acrylic coverage has a thickness between approximately 1.5 mm and 4 mm.

14. The appliance of claim 1, wherein the appliance includes acrylic coverage around canine teeth configured to direct forward movement of anterior teeth during expansion.

15. The appliance of claim 1, wherein the anterior acrylic section covers between 0 mm and 8 mm of the palate measured from a gumline behind the anterior teeth.

16. The appliance of claim 1, wherein the appliance is configured for use in primary dentition, mixed dentition, or permanent dentition.

17. An orthodontic development system, comprising:
a removable airway appliance configured to be positioned in an upper dental arch of a patient;
the appliance including:
an anterior acrylic section configured to contact a palate and lingual surfaces of anterior teeth;
a posterior acrylic section covering occlusal surfaces of posterior teeth;
two expansion screws embedded within the appliance and extending between opposite sides of the appliance;
wherein the expansion screws are adjustable and positioned at selected vertical and lateral angles to control a direction of maxillary development including forward sagittal development, lateral arch expansion, and vertical positional adjustment of anterior teeth; and whereby the appliance promotes forward development of the maxilla and enlargement of an airway space.

18. A method for guiding development of a maxilla and airway of a patient, comprising:
positioning a removable orthodontic airway appliance within an upper dental arch of a patient, the appliance including an anterior acrylic section, a posterior acrylic section, and at least two expansion screws;
orienting the expansion screws at selected vertical and lateral angles;
periodically activating the expansion screws to apply orthodontic forces to the palate and teeth; and
directing maxillary development in a forward direction while optionally increasing arch width and modifying vertical tooth position.

19. The method of claim 18, further comprising adjusting the expansion screws approximately twice per week.

20. The method of claim 18, wherein the appliance is used to increase airway space by repositioning the maxilla and allowing forward positioning of the mandible.
